(19) United States
(12) Patent Application Publication
(10) Pub. No.: US 2016/0136107 A1
(43) Pub. Date: May 19, 2016

(54) STORAGE STABLE TRANSDERMAL PATCH OF ROTIGOTINE

(71) Applicant: MYLAN TECHNOLOGIES, INC., Morgantown, WV (US)

(72) Inventors: Abhijit Deshmukh, Secunderabad (IN); Akhilesh Dixit, Nagpur (IN); Rajesh Kumar Bijay, Hyderabad (IN); Prasanna Kumar, Hyderabad (IN)

(73) Assignee: Mylan Technologies, Inc., Morgantown, WV (US)

(21) Appl. No.: 14/899,957

(22) PCT Filed: Jun. 20, 2014
(86) PCT No.: PCT/US2014/043366

§ 371 Date: Dec. 18, 2015

(30) Foreign Application Priority Data
Jun. 20, 2013 (IN) 1824/DEL/2013

(51) Int. Cl. A61K 9/70; A61K 47/32; A61K 47/14; A61K 31/381; A61K 47/22

(52) U.S. Cl. CPC A61K 9/7053; A61K 31/381; A61K 47/22; A61K 47/14; A61K 47/32

(57) ABSTRACT

Disclosed is a transdermal delivery device comprising a backing layer, a release liner, and an adhesive layer between the backing layer and the release liner, the adhesive layer comprising an active agent solubilized in an adhesive matrix, the adhesive matrix comprising a mixture of biocompatible polymers, wherein the active agent is soluble in at least one of the biocompatible polymers.

### CROSS-REFERENCES TO RELATED APPLICATIONS

The present application claims priority from Indian Patent Application No. 1824/DEL/2013, filed Jun. 20, 2013, the disclosure of which is hereby incorporated herein by reference.

### BACKGROUND OF THE INVENTION

The delivery of drugs through the skin provides many advantages. Primarily, it is a comfortable, convenient and non-invasive way of administering drugs. Moreover, such a means of delivery provides for uninterrupted therapy and a higher degree of control over drug concentrations in the blood.

Transdermal delivery of various active agents and pressure sensitive adhesive matrix patches for transdermal delivery of such active agents are well known in the art of drug delivery. These matrix patches include a pressure sensitive adhesive layer for affixing the patch to the skin and for carrying the active agent and any excipients that are directly incorporated into this adhesive layer into the body. These adhesive matrix patches also typically include an inert backing layer to provide support, and a release liner which covers and protects the adhesive. The release liner is peeled off and discarded before applying the patch to the skin. These patches are distinguished from reservoir patches in that the active agent in a reservoir patch is incorporated in a layer or compartment, with a differing material composition, separate from the pressure sensitive adhesive layer. One type of adhesive commonly used in the adhesive layer is polyisobutylene (“PIB”).

Conventional processes of blending materials in an adhesive blend with a volatile solvent and then removing the solvent by drying cause, it is believed, rapid changes in drug solubility during manufacturing. For example, U.S. Pat. No. 5,508,038 is directed to an adhesive which is useful as an adhesive in transdermal delivery devices. The adhesive comprises mixtures of high molecular weight (“HMW”) and low molecular weight (“LMW”) PIBs in weight ratios of about 5-40 HMW PIB: 95-60 LMW PIB which are substantially free of plasticizers and tackifiers. The system is processed by solvent coating the mixture of PIBs and active agent onto a release liner and then evaporating the solvent from the active agent/PIB adhesive matrix. The process is limited to a batch of solvated PIB and active agent, as well as limited by the necessity and cost of evaporating solvent from the adhesive matrix. Moreover, in the case of highly volatile active agents, it is difficult to evaporate the solvent without also evaporating at least part of the active agent.

The advantage of foregoing the use of solvents lies essentially in the simplification of the coating process. The avoidance of flammable solvents does away with the need for drier units, with their high energy consumption, for the evaporation and recovery of the solvents, and with the need to use explosion-protected units. Hot-melt coating units are compact and permit much higher coating speeds. The technology is environmentally-friendly, with no solvent emissions. Furthermore, no unwanted solvent residues remain in the self-adhesive composition.

### BRIEF SUMMARY OF THE INVENTION

In one aspect of the present invention is a drug-in-adhesive composition comprising an active agent solubilized in an adhesive matrix, wherein the adhesive matrix comprises a mixture of biocompatible polymers, and wherein the active agent is soluble in at least one of the biocompatible polymers. In some embodiments, the active agent is a dopamine agonist. In some embodiments, the dopamine agonist is rotigotine. In some embodiments, an amount of rotigotine ranges from about 5% to about 9% by weight of the composition. In some embodiments, the rotigotine amount ranges from about 6% to about 7.5% by weight of the composition.

In some embodiments, the biocompatible polymers are selected from the group consisting of silicones, natural and synthetic rubbers, polyisobutylene, neoprenes, polybutadienes, polyisoprenes, polysiloxanes, acrylic adhesives including cross-linked and uncross-linked acrylic copolymers, vinyl acetate adhesives, polyacrylates, ethylene vinyl acetates, styrene-isoprene copolymers, polyurethanes, plasticized weight polyether block amide copolymers, and plasticized styrene-rubber block copolymers. In some embodiments, the adhesive matrix comprises polyisobutylene and ethylene vinyl acetate. In some embodiments, a ratio of the polisobutylene to the ethylene vinyl acetate range from about 1.0:0.25 to about 1.0:2.0 In some embodiments, an amount of the ethylene vinyl acetate ranges from about 15% to about 40% by weight of the composition. In some embodiments, the ethylene vinyl acetate amount ranges from about 20% to about 40% by weight of the composition. In some embodiments, the polyisobutylene is present in an amount ranging from about 15% to about 80% by weight of the composition. In some embodiments, the polyisobutylene amount ranges from about 30% to about 50% by weight of the composition.

In some embodiments, the composition further comprises a solubility enhancer. In some embodiments, the solubility enhancer is selected from the group consisting of dimethyl isosorbide, sorbitan monolaurate, and octyl dodecanol. In some embodiments, the solubility enhancer is dimethyl isosorbide. In some embodiments, an amount of the dimethyl isosorbide ranges from about 5% to about 20% by weight of the composition. In some embodiments, the dimethyl isosorbide amount ranges from about 10% to about 15% by weight of the composition.

In some embodiments, the composition further comprises a plasticizer. In some embodiments, the plasticizer is selected from the group consisting of light mineral oil and capric caprylic trglyceride. In some embodiments, the plasticizer is present in an amount ranging from about 5% to about 20% by weight of the composition.

In another aspect of the present invention is a drug-in-adhesive composition comprising an active agent, polyisobutylene, ethylene vinyl acetate, and a solubility enhancer. In another aspect of the present invention is a drug-in-adhesive composition comprising rotigotine, polyisobutylene, ethylene vinyl acetate, and a solubility enhancer. In another aspect of the present invention is a drug-in-adhesive composition comprising rotigotine, polyisobutylene, ethylene vinyl acetate, dimethyl isosorbide, and a plasticizer. In yet another aspect of the present invention is a drug-in-adhesive composition comprising an active agent, polyisobutylene, ethylene vinyl acetate, and a solubility enhancer, wherein the drug-in-adhesive composition is substantially free of solvents.

In another aspect of the present invention is a transdermal delivery device comprising a backing layer, a release liner, and an adhesive layer between the backing layer and the release liner, the adhesive layer comprising an active agent solubilized in an adhesive matrix, the adhesive matrix comprising a mixture of biocompatible polymers, wherein the active agent is soluble in at least one of the biocompatible polymers. In some embodiments, the active agent is a dopamine agonist. In some embodiments, the dopamine agonist is rotigotine. In some embodiments, an amount of rotigotine ranges from about 5% to about 9% by weight of the composition. In some embodiments, the rotigotine amount ranges from about 6% to about 7.5% by weight of the composition.

In some embodiments, the biocompatible polymers are selected from the group consisting of silicones, natural and synthetic rubbers, polyisobutylene, neoprenes, polybutadienes, polyisoprenes, polysiloxanes, acrylic adhesives including cross-linked and uncross-linked acrylic copolymers, vinyl acetate adhesives, polyacrylates, ethylene vinyl acetates, styrene-isoprene copolymers, polyurethanes, plasticized weight polyether block amide copolymers, and plasticized styrene-rubber block copolymers. In some embodiments, the adhesive matrix comprises polyisobutylene and ethylene vinyl acetate. In some embodiments, a ratio of the polisobutylene to the ethylene vinyl acetate range from about 1.0:0.25 to about 1.0:2.0. In some embodiments, an amount of the ethylene vinyl acetate ranges from about 15% to about 40% by weight of the composition. In some embodiments, the ethylene vinyl acetate amount ranges from about 20% to about 40% by weight of the composition. In some embodiments, the polyisobutylene is present in an amount ranging from about 15% to about 80% by weight of the composition. In some embodiments, the polyisobutylene amount ranges from about 30% to about 50% by weight of the composition. In some embodiments, the composition further comprises a solubility enhancer. In some embodiments, the solubility enhancer is selected from the group consisting of dimethyl isosorbide, sorbitan monolaurate, and Octyl dodecanol. In some embodiments, the solubility enhancer is dimethyl isosorbide. In some embodiments, an amount of the dimethyl isosorbide ranges from about 5% to about 20% by weight of the composition. In some embodiments, the dimethyl isosorbide amount ranges from about 10% to about 15% by weight of the composition.

In some embodiments, the composition further comprises a plasticizer. In some embodiments, the plasticizer is selected from the group consisting of light mineral oil and capric caprylic trglyceride. In some embodiments, the plasticizer is present in an amount ranging from about 5% to about 20% by weight of the composition.

In another aspect of the present invention is a method of manufacturing the transdermal delivery device described herein comprising the steps of: (a) heating a mixture of an adhesive material; (b) mixing a solubility enhancer and an active to form a solution; (c) combining the solution and the uniform melt to form a molten mass; and (d) laminating the molten mass to a release liner to form a adhesive matrix layer. In some embodiments, the adhesive matrix comprises polyisobutylene and ethylene vinyl acetate, and are heated to a temperature of between about 140° C. to about 160° C. In some embodiments, the method further comprises the step of applying a backing layer to the adhesive matrix layer.

### BRIEF DESCRIPTION OF THE DRAWINGS

FIG. 1 shows the cumulative permeation of a transdermal device of the present invention compared to a prior art device.

FIG. 2 shows the cumulative permeation of a transdermal device of the present invention compared to a prior art device.

FIG. 3 shows the cumulative permeation of a transdermal device of the present invention compared to a prior art device.

FIG. 4 shows the cumulative permeation of a transdermal device of the present invention compared to a prior art device.

FIG. 5 shows the cumulative permeation of a transdermal device of the present invention compared to a prior art device.

FIG. 6 shows the cumulative permeation of a transdermal device of the present invention compared to a prior art device.

### DETAILED DESCRIPTION

In one aspect of the present invention is a transdermal delivery device comprising a backing layer, a release liner, and an adhesive matrix between the backing layer and release liner, the adhesive matrix comprising a mixture of biocompatible polymers, wherein an active agent is soluble in at least one of the biocompatible polymers. As used herein, “transdermal” means delivery of a drug by passage into and through the skin or mucosal tissue.

In another aspect, the transdermal delivery device is capable of delivering a therapeutically acceptable amount of active agent through the skin of a subject in need thereof for a time period of at least about 24 hours. In some embodiments, the therapeutically acceptable amount is between about 5 μgm/cm2/hr to about 10 μgm/cm2/hr. In other embodiments, the therapeutically acceptable amount is about 8 μgm/cm2/hr. Without wishing to be bound by any particular theory, in some embodiments it is believed that these objectives may be met without the need for antioxidants or stabilizers, or the need to store the transdermal delivery devices in a refrigerated environment.

The backing layer is a flexible substrate which provides a barrier to active drug migration away from the intended direction of drug delivery. Any well-known backing layer which satisfies this purpose can be used in the present invention. Examples of materials from which the backing layer may be composed include polyethylene terephthalate, various nylons, polypropylenes, polyesters, polyester/ethylene-vinyl acetates, metalized polyester films, polyvinylidene chloride, metal films such as aluminum foils, polyvinylidene fluoride films, or mixtures or copolymers thereof.

Other backing layers include ethylene vinyl acetate films laminated to a polyester, ethylene vinyl acetate films laminated to a metalized polyester, Mediflex® 1200 available from Mylan Technologies, Inc., Mediflex® 1501 from Mylan Technologies Inc., Mediflex® 1201 available from Mylan Technologies, Inc., Mediflex® 1502 available from Mylan Technologies, Inc., Dupont polyester type S available from Dupont, Dow BLF® 2050 available from The Dow Chemical Company, 3M™ Scotchpak® 1109 available from 3M, 3M™, Scotchpak® 9723 available from 3M, 3M™ Scotchpak® 9733 available from 3M, 3M™ Scotchpak® 9735 available from 3M and 3M™ Scotchpak® 9730 available from 3M.

Silicone coated polyethylene backings, such as Mediflex® 1000 coated with a silicone layer, 3M™ Cotran® 9722 coated with a silicone layer, and 3M™ Cotran™ 9720 coated with a silicone layer, preserve the amorphous form of the drug in the adhesive matrix. Similarly, silicone coated polyester backings, such as Mediflex® 1200 coated with a silicone layer, also preserves the amorphous form of drug in adhesive.

In some embodiments, the backing layer may be the same size as the adhesive layer. In other embodiments, the backing layer may be oversized as compared with the adhesive layer, i.e. the backing layer may be larger than the adhesive layer. In yet other embodiments, the backing layer may range from about 0.01 mm to at least 10 mm larger than the adhesive matrix layer, preferably ranging from about 0.05 mm to about 5 mm larger than the adhesive matrix layer, and most preferably ranging from about 0.1 mm to about 3 mm larger than the adhesive matrix layer. Without wishing to be bound by any particular theory, it is believed that the use of an oversized backing layer helps prevent the adhesive matrix from becoming distorted or relaxing during the handling and/or shipping processes.

Adjacent to the backing layer is an adhesive matrix layer. The adhesive matrix is comprised of a mixture of biocompatible polymers such that either (1) an active agent is soluble in at least one of the biocompatible polymers; or (2) the polymers are miscible in each other.

The adhesive matrix may be comprised of any mixture of biocompatible polymers or polymeric materials known in the art. One skilled in the art will be able to select an appropriate mixture of biocompatible polymers such that the active agent is solubilized within the adhesive matrix.

The biocompatible polymers may be selected from silicones, natural and synthetic rubbers, polyisobutylene, neoprenes, polybutadienes, polyisoprenes, polysiloxanes, acrylic adhesives including cross-linked and uncross-linked acrylic copolymers, vinyl acetate adhesives, polyacrylates, ethylene vinyl acetates (“EVA”), styrene-isoprene copolymers, polyurethanes, plasticized weight polyether block amide copolymers, plasticized styrene-rubber block copolymers, and mixtures thereof.

In some embodiments, the adhesive matrix comprises between about 15% to about 80% by total weight of polymer. In other embodiments, the adhesive matrix comprises between about 35% and about 75% by total weight of polymer.

In some embodiments, the adhesive matrix is comprised of polyisobutylene, or a mixture of polyisobutylenes having differing molecular weights (collectively referred to herein as “polyisobutylene”), and at least one other biocompatible polymer in which the active agent is soluble.

In other embodiments, the adhesive matrix comprises polyisobutylene and an ethylene vinyl acetate. In some embodiments, the amount of polyisobutylene ranges from between about 15% and about 80% by weight total weight of the adhesive matrix while an amount of ethylene vinyl acetate ranges from between about 15% to about 40% by total weight of the adhesive matrix. In other embodiments, the amount of polyisobutylene ranges from between about 30% and about 50% by weight total weight of the adhesive matrix while an amount of ethylene vinyl acetate ranges from between about 20% to about 40% by total weight of the adhesive matrix.

In some embodiments, the ratio of PIB to EVA is about 1.0:0.25 to about 1.0:2.0.

Without wishing to be bound by any particular theory, it is believed that ethylene vinyl acetate (with a vinyl acetate content of about 40%) is suitable and miscible in a polyisobutylene matrix. It is also believed that ethylene vinyl acetate may be used over a concentration ranging from about 0% to about 40% by total weight of the adhesive matrix. Those of ordinary skill in the art will be able to select an appropriate amount of ethylene vinyl acetate to solubilize an active agent and to prevent or mitigate the crystallization of the active agent.

The adhesive matrix comprises at least one active agent. As used herein, the term “active agent” is used to describe the principal active ingredient of the transdermal delivery device, which is a biologically active compound or mixture of compounds that has a therapeutic, prophylactic and/or physiological effect on the wearer of the device.

Non-limiting examples of active agents include anti-inflammatory substances, dopamine agonists, opioid receptor antagonists, anticholinergics, coronary dilators, cerebal dilators, peripheral vasodilators, alpha-adrenergic blockers, anti-infectives, psychotropics, anti-manics, stimulants, anti-histamines, decongestants, gastro-intestinal sedatives, anti-anginal drugs, vasodilators, anti-arrhythmics, anti-hypertensive drugs, vasoconstrictors, migraine treatments, anti-coagulants and anti-thrombotic drugs, analgesics, anti-pyretics, hypnotics, sedatives, anti-emetics, anti-nauseants, anti-convulsants, neuromuscular drugs, hyper- and hypoglycemic agents, thyroid and anti-thyroid preparations, diuretics, anti-spasmodics, anti-emetic, uterine relaxants, anti-obesity drugs, anabolic drugs, erythropoietic drugs, anti-asthmatics, bronchodilators, expectorants, mucolytics, anti-uricemic drugs, narcotics, anti-depressants, agents for treating alcohol abuse or dependence and the like.

In some embodiments of the present invention, the active pharmaceutical ingredient is selected from compounds including testosterone, tulobuterol, buprenorphine, dextroamphetamine, flurbiprofen, rotigotine, respiridone, dexketoprofen, ketoprofen, diclofenac, rivastigmine, granisetron, nicotine, methylphenidate, lidocaine, tetracaine, prilocaine and bupivicaine, hydrocortisone, cortisone, dexamethasone, prednisolone, prednisone, halcinonide, methylprednisolone, flurocortsone, corticosterone, paramethasone, ibuprofen, napoxen fenoprofen, fenbufen, indoprofen, salicylic acid, methylslicyate, sulindac, mefenamic acid, piroxicam, indonisilone, tolmetin, propranolol, isosorbide dinitrate, isosorbide mononitrates, diltiazem, nifedipine, procinamide, methyltestosterone, fluoxmesterone, 1-B estradiol valerate, equilin, mestranol estrone, estriol, diethylstilbestrol, progesterone, 19-norprogesterone, norethindrone, melengetrol chloradinone, ethisterone, medroxyprogesterone acetate, hydroxyprogesterone caproate, noethynodrel, dimethisterone, ethinylestrenol, norgestrel, megestrolacetate, ethinodiol diacetate, chloral hydrate, benzdiazepines, naloxone, haloperidol, pentobarbitol, phenobarbitol, secobarbitol, codeine, dibucaine, benzocaine, fentanyl analogs, diphenhydramine, triprolidine, chlorcyclizine, promethazine, cyclizine, chlorprenaline, terrenadine, phenylpropanolamine, chlorpheniramine, pilocarpine, atropine, methantheline, papverine, cinnmedrine, methscopolamine, naltrexone, isocaboxazide, phenelzine, imipramine, amitrptyline, trimepramine, dozepin, desipramine, nortriptyline, protriptyline, amoxapine, maprotiline, tamoxifen, tetracycline, chloramphenicol, sulfacetamide, sulfadiazine, sulfamerazine, sulfoxazole, idoxuridine, erythromycin, antazoline, metapyriene, pyrilamine, aspirin, salicylamide, dihdroergotamine, pizotyline, reserpine, chlorpromazine, antianxiety benzodiazepines, helperidol loxapine, molindone, thiothixene, pimozide, quetiapine fumarate, olanzapine, loratadine, desloratadine, dexmethylphenidate, and phenothiazine derivatives. In yet other embodiments, the active pharmaceutical ingredient is selected from compounds including fentanyl, scopolamine, nitroglycerin, clonidine, 17-β estradiol, ethinyl estradiol, norelgestromin (and other hormones for birth control), selegiline, methylphenidate, oxybutynin, nicotine, diclophenac, or rivastigamine. Of course, any of these active pharmaceutical ingredients may be in the form of a salt, solvate, or hydrate or mixtures thereof. Also contemplated are derivatives or analogs of these compounds. In some embodiments, two or more active agents (in any form) may be combined.

In some embodiments, the active agent is a non-ergotinic dopamine agonist.

In other embodiments, the active agent is rotigotine ((S)-6-[propyl(2-thiophen-2-ylethyl)amino]-5,6,7,8-tetrahydronaphthalen-1-ol). The rotigotine may be present as its free base, as a salt, a hydrate, or mixtures thereof.

In other embodiments, the active agent is any agent having a low melting point.

In some embodiments, the active agent is generally present in an amount ranging from about 5% to about 9% by weight of the adhesive matrix layer. In other embodiments, the active agent is generally present in an amount ranging from about 6% to about 7.5% by weight of the adhesive matrix layer.

In some embodiments, the adhesive matrix comprises rotigotine, polyisobutylene (or a mixture of different molecular weight polisobutylenes), and a biocompatible polymer in which the rotigotine is soluble. In other embodiments, the adhesive matrix comprises rotigotine, polyisobutylene, and ethylene vinyl acetate. In other embodiments, the amount of polyisobutylene ranges from between about 30% and about 60% by weight total weight of the adhesive matrix, an amount of ethylene vinyl acetate ranges from between about 10% to about 50% by total weight of the adhesive matrix, and an amount of rotigotine ranges from about 5% to about 9% by total weight of the adhesive matrix.

The adhesive matrix layer may contain one or more additives selected from solubility enhancers, tackifiers, cohesive enhancers, permeation enhancers, crystal growth inhibitors, plasticizers, antioxidants, flux enhancers, penetration enhancers, and/or other pharmaceutically acceptable additives or excipients. In some embodiments, the additives are generally present in the composition in an amount ranging from about 1% to about 50% by weight of the adhesive matrix layer. In other embodiments, the additives are generally present in the composition in an amount ranging from about 5% to about 30% by weight of the adhesive matrix layer.

In some embodiments, the adhesive matrix comprises a solubility enhancer. Suitable solubility enhancers include sorbitan monolaurate, dimethyl isosorbide and octyl dodecanol. In other embodiments, the solubility enhancer is dimethyl isosorbide. Without wishing to be bound by any particular theory, it is believed that the combination of dimethyl isosorbide and ethylene vinyl acetate in an adhesive matrix provides for stability and/or solubility of the active agent while maintaining a sufficient thermodynamic activity of the active agent to provide for therapeutic rates of delivery through the skin.

In some embodiments, the solubility enhancer is present in an amount ranging from about 5% to about 20% by weight of the adhesive matrix. In other embodiments, the solubility enhancer is present in an amount ranging from about 10% to about 20% by weight of the adhesive matrix.

In some embodiments, the adhesive matrix comprises a plasticizer. Suitable plasticizers include Capric caprylic triglyceride (GTCC), mineral oil and Polybutene. In other embodiments, the plasticizer is Capric caprylic triglyceride (Crodomol GTCC).

In some embodiments, the plasticizer is present in an amount ranging from about 0% to about 25% by weight of the adhesive matrix. In other embodiments, the plasticizer is present in an amount ranging from about 5% to about 15% by weight of the adhesive matrix.

Adjacent to the adhesive matrix layer is a release liner. Release liners well known in the art can be used in the present invention. Examples of materials from which the release liner may be composed include polyethylene terephthalate/silicone (i.e. polydimethyl siloxane) (“PET/SI”), polyethylene terephthalate/aluminized polyester coated with silicone (i.e. polydimethyl siloxane) (“PET/MET/SI”), polyester or polyurethane liners with a silicone coating, polyester or polyurethane liners with a fluorosilicone coating, or polyester or polyurethane liners with a silicon coating. Specific release liners include Medirelease® 2249, Medirelease® 2226, Medirelease® 2500, 3M™ Scotchpak® 1020, 3M™ Scotchpack® 1022, 3M™ Scotchpak® 9741, 3M™ Scotchpak® 9742, 3M™ Scotchpak® 9744, CPFilms Inc. Clearsil® UV5A and CPFilms Inc., Clearsil® UV510, CPFilms Inc. Sil® UV5A and CPFilms Inc. Sil® UV510.

In some embodiments, the release liner may be the same size as the adhesive matrix layer and/or may be the same size as the backing layer. In other embodiments, the release liner may be larger than the adhesive matrix layer and/or may be larger than the backing layer. In yet other embodiments, the release liner may range from about 0.1 mm to at least about 20 mm larger than the margin of a backing layer or an adhesive matrix layer, preferably ranging from about 0.5 mm to about 10 mm larger than the backing layer or adhesive matrix layer, and most preferably ranging from about 1 mm to about 5 mm larger than the backing layer or adhesive matrix layer. It is believed that the use of an oversized release liner facilitates its removal by the user prior to application to the skin and may also help prevent the adhesive matrix from becoming distorted or relaxing during the handling and shipping processes.

The transdermal delivery devices of the present invention are capable of providing a therapeutically acceptable release of active agent over a time period of between about 15 and 24 hours. In some embodiments, the devices are able to provide a cumulative flux between about 5 μg/sq.com to about 30 μg/sq.com over a period of between about 3 hours and about 18 hours.

The present invention also is directed to a substantially solvent-free process for manufacturing a transdermal delivery device or patch comprising a backing layer, a release liner, and an adhesive matrix layer. As used herein, the term “solvent free” means that the manufacturing process does not use solvents, or uses solvents (either added or present in the starting materials) in an amount of less than about 85% of the quantities normally used in the manufacture of a transdermal delivery device or patch. It is believed that the use of a hot-melt coating technique eliminates or mitigates the need for incorporating and then removing solvents during manufacture. It is believed that the hot-melt process does not require a solvent so it avoids the risk of super-saturation during manufacturing or uncontrolled crystallization during storage.

In general, the adhesive matrix layer and the transdermal delivery device comprising it is made according to a hot melt process, as that process is known in the art. In some embodiments, the device is made substantially without the use of solvents. Essentially, the biocompatible polymers are weighed, along with any additives, and heated together to form a uniform melt. The components are generally heated at a temperature between about 110° C. to about 150° C.

Separately, the active agent and additives are combined and mixed, sonicated or blended until a solution is obtained.

The uniform melt and solution are then mixed together and optionally heated to form a molten mass. If heated, the components are generally heated at a temperature between about 120° C. to about 200° C.

The molten mass is then transferred to a release liner. A backing layer is then added.

Examples 1, 2, and 3 (shown in Table 1 below) each illustrate certain embodiments of a drug-in-adhesive composition comprising rotigotine as an active agent, polyisobutylene (Oppanol B 12), ethylene vinyl acete (Elvax 40W), dimethyl isosorbide (Arlasolve DMI), and capric caprylic triglyceride (Crodamol GTCC). Each of the drug-in-adhesive compositions, when incorporated into a transdermal delivery device, are capable of delivering therapeutically acceptable amounts of active agent for a time period of about 24 hours.

TABLE 1
Examples 1, 2, and 3 illustrate different compositions having
differing amounts of active agent.
Batches with combination of EVA and PIB adhesive by
Hot melt coating process

| Composition (% w/w) | Example 1 | Example 2 | Example 3 |
|---|---|---|---|
| Rotigotine | 6.5 | 7.0 | 7.5 |
| Oppanol B 12 | 46.0 | 45.75 | 45.5 |
| Arlasolve DMI | 15.0 | 15.0 | 15.0 |
| Crodamol GTCC | 10.0 | 10.0 | 10.0 |
| Elvax 40 W | 22.50 | 22.25 | 22.0 |

Process for the production of the compositions of Examples 1, 2, and 3 and the incorporation of the drug-in-adhesive compositions into a transdermal delivery device are as follows:

1. Weigh a batch quantity of Oppanol B12, Elvax 40 W and Crodamol GTCC in a beaker and heat to about 140 to about 160° C. for about 20 to about 30 minutes in an oil bath with an intermittent mixing to form uniform melt.

2. Weigh Arlasolve DMI in a beaker and add active agent into it and sonicate for about 10 to about 15 min until a clear solution obtained.

3. Add step 2 into step 1 and mix to form uniform melt. Maintain temperature at about 110 to about 140° C.

4. Transfer the molten mass to the reservoir of Hot Melt coater Laminator 1000 and set the temperature of the upper roller, bottom roller and reservoir plate at about 250° F., respectively.

5. Hold the molten mass in reservoir for about 10 minutes and coat on a release liner at target thickness/gsm.

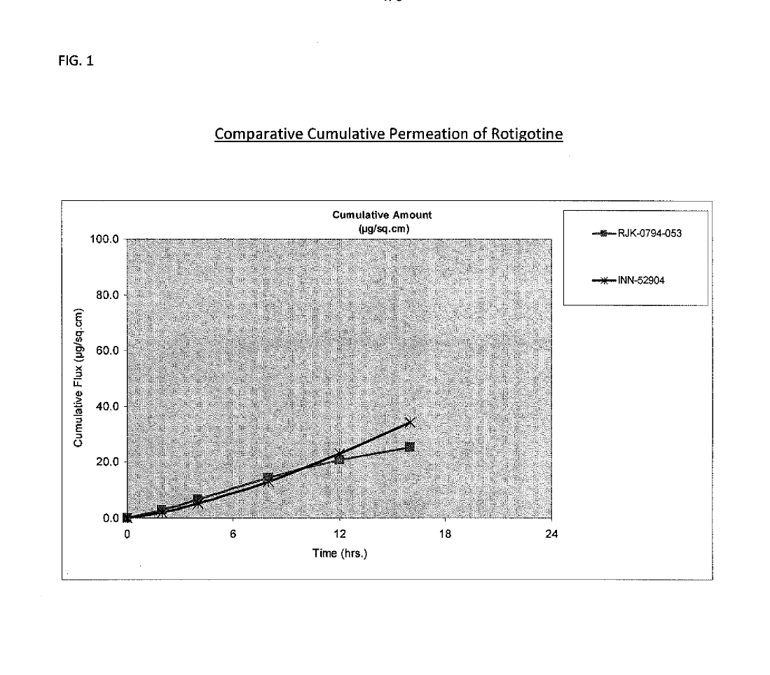

FIG. 1 shows the cumulative permeation of rotigotine from a transdermal delivery device according to Example 1. The cumulative permeation of the device of example 1 is about the same as compared with INNO-52901 (a prior art transdermal rotigotine delivery system) over a period of about 18 hours.

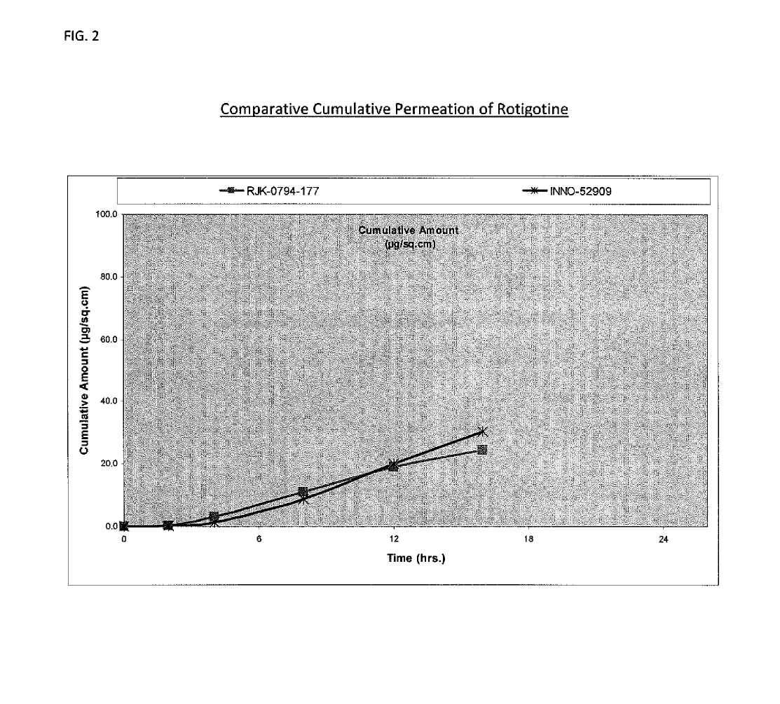

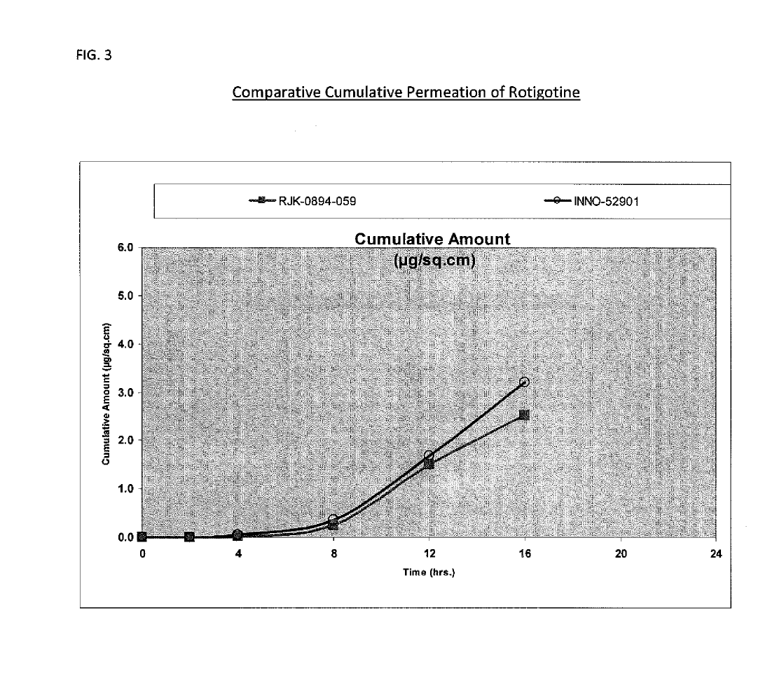

FIGS. 2 and 3 shows the cumulative permeation of rotigotine from a transdermal delivery device according to Example 3. The cumulative permeation of the device of example 3 is about the same as compared with INNO-52901 (a prior art transdermal rotigotine delivery system) over a period of between about 16 and 18 hours.

TABLE 2
Examples 4, 5, and 6 illustrate different compositions having
differing amounts of active agent.
Batches with combination of EVA and PIB adhesive by
Hot melt coating process

| Composition (% w/w) | Example 4 | Example 5 | Example 6 |
|---|---|---|---|
| Rotigotine | 6.0 | 7.0 | 7.0 |
| Oppanol B 10 | 56.5 | 55.5 | 58.0 |
| Arlasolve DMI | 15.0 | 15.0 | 15.0 |
| Crodamol GTCC | 10.0 | 15.0 | 10.0 |
| Elvax 40 W | 13.5 | 12.5 | 10.0 |

Process for the production of the compositions of Examples 4, 5, and 6 and the incorporation of the drug-in-adhesive compositions into a transdermal delivery device are as follows:

1. Weigh a batch quantity of Oppanol B10, Elvax 40 W and Crodamol GTCC in a beaker and heat to about 110° C. for about 20 to 30 minutes in an oil bath with an intermittent mixing to a form uniform melt.

2. Weighed Arlasolve DMI in a beaker and add drug into it and sonicate for 10-15 min till clear solution obtained.

3. Add step 2 into step 1 and mix to form a uniform melt. Maintain temperature at about 110° C.

4. Transfer the molten mass to the reservoir of Hot Melt coater Laminator 1000 and set temp of upper roller, bottom roller and reservoir plate at about 215° F., respectively.

5. Hold the molten mass in a reservoir for about 10 minutes and coat on release liner at target thickness/gsm.

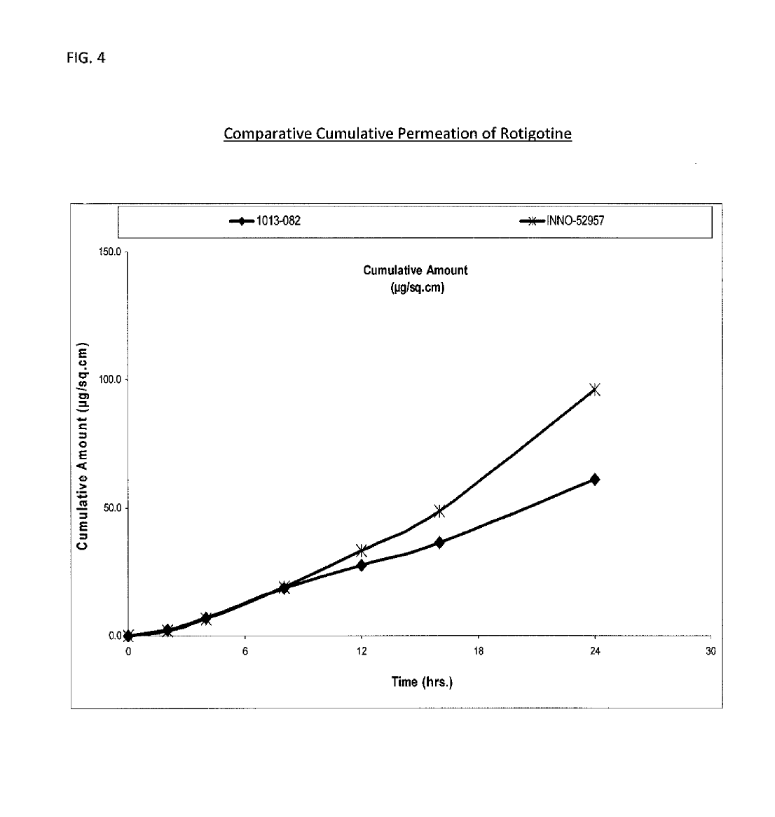

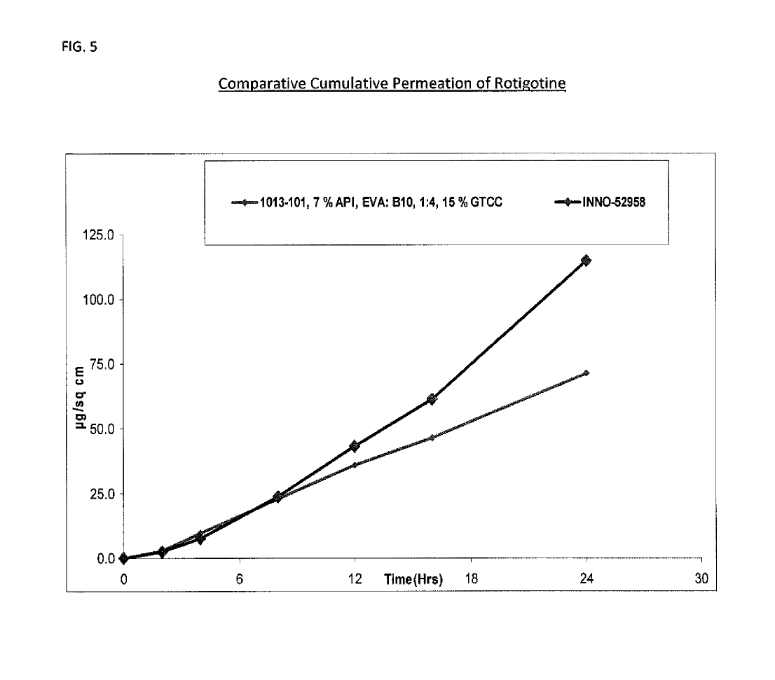

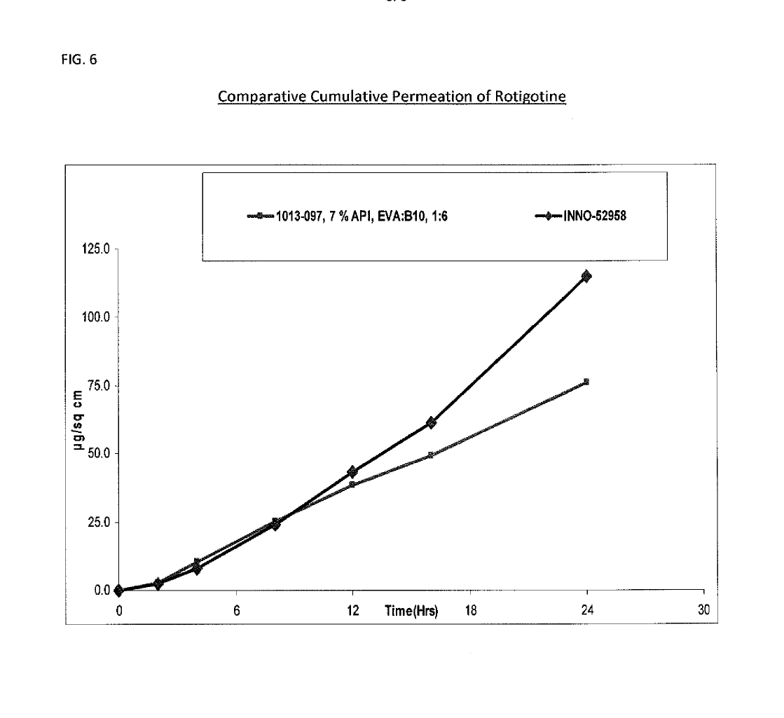

FIGS. 4, 5, and 6 show the cumulative permeation of rotigotine from a transdermal delivery device according to Examples 4, 5, and 6. The cumulative permeation of the device of Examples 4, 5, and 6 is less than the cumulative permeation of a prior art transdermal system, INNO-52901 or INNO-52958 over a period of between about 12 and 24 hours.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1-43. (canceled)

44. A composition for application to the skin comprising a pharmaceutical active agent solubilized in an adhesive matrix, said adhesive matrix comprising a mixture of biocompatible polymers, wherein said pharmaceutical active agent is soluble in each of said mixture of said biocompatible polymers, without the addition of a solvent whereby a solvent-free process can be utilized to produce said adhesive matrix.

45. The composition of claim 44, wherein said active agent is a dopamine agonist.

46. The composition of claim 45 wherein said dopamine agonist is rotigotine.

47. The composition of claim 46, wherein an amount of said rotigotine ranges from 5% to 9% by weight of said composition.

48. The composition of claim 47 wherein said amount ranges from 6% to 7.5% by weight of said composition.

49. The composition of claim 44, wherein said biocompatible polymers are selected from the group consisting of silicones, natural and synthetic rubbers, polyisobutylene, neoprenes, polybutadienes, polyisoprenes, polysiloxanes, acrylic adhesives including cross-linked and uncross-linked acrylic copolymers, vinyl acetate adhesives, polyacrylates, ethylene vinyl acetates, styrene-isoprene copolymers, polyurethanes, plasticized weight polyether block amide copolymers, and plasticized styrene-rubber block copolymers.

50. The composition of claim 44, wherein said adhesive matrix comprises polyisobutylene and ethylene vinyl acetate.

51. The composition of claim 50, wherein a ratio of said polisobutylene to said ethylene vinyl acetate ranges from about 1.0:0.25 to about 1.0:2.0.

52. The composition of claim 50, wherein an amount of said ethylene vinyl acetate ranges from 15% to 40% by weight of said composition.

53. The composition of claim 52, wherein said amount ranges from 20% to 40% by weight of said composition.

54. The composition of claim 50, wherein said polyisobutylene is present in an amount ranging from 15% to 80% by weight of said composition.

55. The composition of claim 54, wherein said amount ranges from 30% to 50% by weight of said composition.

56. The composition of claim 44, further comprising a solubility enhancer.

57. The composition of claim 56, wherein said solubility enhancer is selected from the group consisting of dimethyl isosorbide, sorbitan monolaurate, and Octyl dodecanol.

58. The composition of claim 57, wherein said solubility enhancer is dimethyl isosorbide.

59. The composition of claim 58, wherein an amount of said dimethyl isosorbide ranges from 5% to 20% by weight of said composition.

60. The composition of claim 59, wherein said amount ranges from 10% to 15% by weight of said composition.

61. The composition of claim 44, further comprising a plasticizer.

62. The composition of claim 61, wherein said plasticizer is selected from the group consisting of light mineral oil and capric caprylic trglyceride.

63. The composition of claim 62, wherein said plasticizer is present in an amount ranging from 5% to 20% by weight of said composition.

64. A transdermal delivery device for application to the skin comprising a backing layer, a release liner, and an adhesive layer between said backing layer and said release liner, said adhesive layer comprising a pharmaceutical active agent solubilized in an adhesive matrix, said adhesive matrix comprising a mixture of biocompatible polymers, wherein said pharmaceutical active agent is soluble in each of said mixture of said biocompatible polymers without the addition of a solvent, whereby a solvent-free process can be utilized to produce said adhesive matrix.

65. The composition of claim 64, wherein said active agent is a dopamine agonist.

66. The composition of claim 65, wherein said dopamine agonist is rotigotine.

67. The composition of claim 66, wherein an amount of said rotigotine ranges from 5% to 9% by weight of said composition.

68. The composition of claim 67, wherein said amount ranges from 6% to 7.5% by weight of said composition.

69. The composition of claim 64, wherein said biocompatible polymers are selected from the group consisting of silicones, natural and synthetic rubbers, polyisobutylene, neoprenes, polybutadienes, polyisoprenes, polysiloxanes, acrylic adhesives including cross-linked and uncross-linked acrylic copolymers, vinyl acetate adhesives, polyacrylates, ethylene vinyl acetates, styrene-isoprene copolymers, polyurethanes, plasticized weight polyether block amide copolymers, and plasticized styrene-rubber block copolymers.

70. The composition of claim 64, wherein said adhesive matrix comprises polyisobutylene and ethylene vinyl acetate.

71. The composition of claim 70, wherein a ratio of said polisobutylene to said ethylene vinyl acetate ranges from 1.0:0.25 to 1.0:2.0

72. The composition of claim 70, wherein an amount of said ethylene vinyl acetate ranges from 15% to 40% by weight of said composition.

73. The composition of claim 72, wherein said amount ranges from 20% to 40% by weight of said composition.

74. The composition of claim 70, wherein said polyisobutylene is present in an amount ranging from 15% to 80% by weight of said composition.

75. The composition of claim 74, wherein said amount ranges from 30% to 50% by weight of said composition.

76. The composition of claim 64, further comprising a solubility enhancer.

77. The composition of claim 76, wherein said solubility enhancer is selected from the group consisting of dimethyl isosorbide, sorbitan monolaurate, and Octyl dodecanol.

78. The composition of claim 77, wherein said solubility enhancer is dimethyl isosorbide.

79. The composition of claim 78, wherein an amount of said dimethyl isosorbide ranges from 5% to 20% by weight of said composition.

80. The composition of claim 79, wherein said amount ranges from 10% to 15% by weight of said composition.

81. The composition of claim 64, further comprising a plasticizer.

82. The composition of claim 81, wherein said plasticizer is selected from the group consisting of light mineral oil and capric caprylic trglyceride.

83. The composition of claim 82, wherein said plasticizer is present in an amount ranging from 5% to 20% by weight of said composition.

84. A method of manufacturing the transdermal delivery device of claim 64, comprising the steps of:
a. heating a mixture of an adhesive material to form a uniform melt;
b. mixing a solubility enhancer and a pharmaceutical active agent to form a solution;
c. combining said solution and said uniform melt to form a molten mass without the addition of a solvent whereby a solvent-free process can be utilized to produce said molten mass; and
d. laminating said molten mass to a release liner to form an adhesive matrix layer.

85. The method of claim 84, wherein said adhesive material comprises polyisobutylene and ethylene vinyl acetate, and said adhesive material is heated to a temperature of between about 140° C. to about 160° C.

86. The method of claim 85, further comprising the step of applying a backing layer to said adhesive matrix layer.
